(19) United States
(12) Patent Application Publication
(10) Pub. No.: US 2023/0181773 A1
(43) Pub. Date: Jun. 15, 2023

(54) VISUALIZABLE RADIOACTIVE CARBON MICROSPHERE (CMS), PREPARATION METHOD, AND USE THEREOF

(71) Applicant: CHENGDU NEW RADIOMEDICINE TECHNOLOGY CO., LTD., Chengdu (CN)

(72) Inventors: Xiaosheng ZHAO, Ganzhou (CN); Jun ZHANG, Guangyuan (CN); Chixiang ZHANG, Chengdu (CN); Jing LU, Chongqing (CN); Qiangqiang LIU, Chengdu (CN); Xianjin LUO, Zhaotong (CN); Wenliang MA, Chengdu (CN); Qiang GE, Chengdu (CN); Jiming CAI, Chengdu (CN)

(73) Assignee: CHENGDU NEW RADIOMEDICINE TECHNOLOGY CO., LTD., Chengdu (CN)

(21) Appl. No.: 17/916,080

(22) PCT Filed: Apr. 23, 2021
(86) PCT No.: PCT/CN2021/089200

§ 371 Date: Sep. 30, 2022

(30) Foreign Application Priority Data
Aug. 26, 2020 (CN) 202010867997.6

(51) Int. Cl. A61K 51/12; A61K 51/04

(52) U.S. Cl. CPC A61K 51/1217; A61K 51/1251; A61K 51/0402

(57) ABSTRACT

A visualizable radioactive carbon microsphere (CMS) suspension, a preparation method, and a use thereof are provided. Every 1 mL of the visualizable radioactive CMS suspension includes: CMS: 10 mg to 500 mg; a therapeutic radionuclide with an activity of 5 mCi to 500 mCi; an imaging radionuclide with an activity of 0.1 mCi to 100 mCi; a small organic molecule: 0 mg to 100 mg; and a first solution: 0.1 mL to 1.0 mL. The preparation method mainly includes a process of allowing the CMS to adsorb the small organic molecule, the therapeutic radionuclide, and the imaging zirconium [⁸⁹Zr]. The visualizable radioactive CMS suspension can realize both local radiotherapy and real-time imaging of a solid tumor lesion, and thus achieves the visualized treatment of a tumor, which provides a new radioactive CMS product that integrates diagnosis and treatment.

### CROSS REFERENCE TO THE RELATED APPLICATIONS

This application is the national phase entry of International Application No. PCT/CN2021/089200, filed on Apr. 23, 2021, which is based upon and claims priority to Chinese Patent Application No. 202010867997.6, filed on Aug. 26, 2020, the entire contents of which are incorporated herein by reference.

### TECHNICAL FIELD

The present disclosure relates to the technical field of medicine, and in particular to a visualizable radioactive carbon microsphere (CMS) suspension, a preparation method, and a use thereof.

### BACKGROUND

In recent years, local minimally invasive radiotherapy has played an increasingly important role in clinical treatment. In particular, the local minimally invasive radiotherapy has been more and more widely used in the treatment of unresectable solid tumors, such as liver cancer, prostate cancer, and kidney cancer.

Currently, the radioactive microsphere products clinically used in the local minimally invasive radiotherapy for advanced liver cancer mainly include yttrium [⁹⁰Y] resin microspheres SIR-Spheres® (Sirtex Medical Limited, Australia) and yttrium [⁹⁰Y] glass microspheres TheraSphere® (BTG, UK). However, because the radioactive yttrium [⁹⁰Y] nuclide is a pure β nuclide and can only generate y rays for imaging through bremsstrahlung radiation, only blurred images (as shown in FIG. 1) can be provided in single photon emission computed tomography (SPECT) and positron emission tomography (PET), such that the in vivo distribution data of the radioactive microspheres and the changes of lesion locations cannot be accurately obtained. Therefore, both yttrium [⁹⁰Y] resin microspheres and yttrium [⁹⁰Y] glass microspheres can only provide low-quality SPECT and PET images, which are not sufficiently clear for diagnosis and treatment. When such radioactive microspheres enter a lesion location, the following assessments can only be conducted by other imaging techniques: 1. Whether a drug enters a target location accurately. 2. Whether the radioactive microspheres move to other tissues and organs during the treatment process. 3. The therapeutic effect of the treatment site. However, these imaging techniques can only achieve short-term imaging and require the use of a contrast agent in addition to the therapeutic drug, which causes at least the following two problems: 1. The contrast agent needs to be administered separately from the therapeutic drug for imaging, which brings great inconvenience during clinical use. 2. The in vivo distribution and metabolism of the contrast agent are inconsistent with those of the radioactive microspheres, so that and the location of the radioactive microspheres cannot be accurately monitored for a long time.

Therefore, to realize the visualization of the entire drug delivery process, accurately monitor the in vivo distribution of radiotherapeutic microspheres during a treatment process, and efficiently evaluate a therapeutic effect, the realization of high-quality visualization of radiotherapeutic microspheres is an urgent problem that must be solved in the art.

### SUMMARY

To solve the problems in the background art and realize the visualized treatment of a radioactive CMS product in a tumor treatment process, the present disclosure provides a radioactive CMS suspension that is visible on images and a preparation method thereof. The CMS suspension is clearly visible in PET and can be quantitatively analyzed, such that the visualization of a radiotherapy process in a solid tumor can be achieved without the aid of other contrast agents.

According to a technical solution provided by the present disclosure, the visualizable radioactive CMS suspension is obtained by dispersing CMSs with prominent biocompatibility in a special solution and a therapeutic radionuclide and zirconium [⁸⁹Zr] are loaded onto the CMSs by being added to the special solution, and radioactive CMSs in the suspension can emit high-energy β rays for solid tumor therapy and PET imaging.

To achieve the above objective, the present disclosure adopts a first technical solution as follows:

A visualizable radioactive CMS suspension is provided, where every 1 mL of the visualizable radioactive CMS suspension includes: CMS: 10 mg to 500 mg; a therapeutic radionuclide with an activity of 5 mCi to 500 mCi; an imaging radionuclide with an activity of 0.1 mCi to 100 mCi; a small organic molecule: 0 mg to 100 mg; and a first solution: 0.1 mL to Further, the CMS may be a spherical or non-spherical carbon material rich in micropores and mesopores that is prepared by any method and may have a diameter of 0.05 μm to 1,000 μm and preferably 20 μm to 60 μm. Visualizable CMS products prepared from CMSs with different particle sizes can be used for the visualized treatment of different solid tumor lesions due to different administration routes and different distributions in tissues, organs, and lesions.

Further, the small organic molecule may be 5-sulfosalicylic acid (5-SSA), 5-nitrosalicylic acid (5-NSA), or a small molecule with a similar structure that is obtained through simple chemical modification.

Further, the therapeutic radionuclide may be any one selected from the group consisting of yttrium [⁹⁰Y], lutetium [¹⁷⁷Lu], holmium [¹⁶⁶Ho], samarium [¹⁵³Sm], and isotopes thereof, and the imaging radionuclide may be zirconium [⁸⁹Zr].

Further, the first solution may include, but is not limited to, an ethanol solution, a polyethylene glycol (PEG) solution, and a glycerol solution; a water-soluble saccharide solution, such as a glucose solution, a dextran solution, and a dextran solution; a water-soluble cellulose solution, such as a sodium carboxymethyl cellulose (CMC-Na) solution, a sodium carboxyethyl cellulose (CEC-Na) solution, and a hydroxypropyl cellulose (HPC) solution; a water-soluble starch solution, such as a hydroxyethyl starch (HES) solution and a sodium carboxymethyl starch (CMS-Na) solution; and another solution of a water-soluble small molecule or polymer with a similar structure.

Further, the therapeutic radionuclide and zirconium [⁸⁹Zr] may be water-soluble therapeutic radionuclide and zirconium [⁸⁹Zr] that are in different chemical forms and are prepared by a generator, a nuclear reactor, or any other common preparation method. A therapeutic radionuclide solution may have a radioactivity of 10 mCi to 100 Ci, and a radioactive zirconium [⁸⁹Zr] solution may have a radioactivity of 0.1 mCi to 10 Ci.

The present disclosure adopts a second technical solution as follows:

A preparation method of the visualizable radioactive CMS suspension is provided, including the following steps:

thoroughly mixing a therapeutic radionuclide solution with a small organic molecule aqueous solution of a first pH to obtain a mixed solution;

allowing the CMS to adsorb the therapeutic radionuclide in the mixed solution;

mixing the CMS adsorbing the therapeutic radionuclide with a sodium phosphate solution to allow a reaction, washing, and conducting solid-liquid separation (SLS) to obtain a first intermediate;

thoroughly mixing the first intermediate with the small organic molecule aqueous solution of the first pH to obtain a first intermediate solution;

adding a radioactive zirconium [⁸⁹Zr] ion solution of a second pH to the first intermediate solution to obtain a second intermediate; and

adding the first solution to the second intermediate, and thoroughly mixing and sterilizing the resulting mixture.

The present disclosure adopts a third technical solution as follows:

A preparation method of the visualizable radioactive CMS suspension is provided, including the following steps:

allowing the CMS to adsorb the small organic molecule in a small organic molecule aqueous solution of a first pH;

allowing the CMS adsorbing the small organic molecule to adsorb the radioactive zirconium [⁸⁹Zr] to obtain a third intermediate;

allowing the third intermediate to adsorb the therapeutic radionuclide in a mixed solution of a therapeutic radionuclide ion solution and a small organic molecule aqueous solution;

mixing the third intermediate adsorbing the therapeutic radionuclide with a sodium phosphate solution to allow a reaction, washing, and conducting SLS to obtain a fourth intermediate; and

adding the first solution to the fourth intermediate, and thoroughly mixing and sterilizing the resulting mixture.

Further, the first pH may be 1 to 14 and preferably 3.5 to 6.5; the second pH may be 1 to 14 and preferably 3.5 to 7.5.

The present disclosure also provides a use of the visualizable radioactive CMS suspension described above in the preparation of a drug for the visualized treatment of a tumor, where the tumor includes, but is not limited to, liver cancer, pancreatic cancer, kidney cancer, breast cancer, thyroid cancer, bowel solid tumor, and bone tumor.

Further, the visualized treatment of the tumor may refer to the realization of both local radiotherapy and real-time imaging of a tumor lesion, and the imaging may be conducted by positron emission tomography/computed tomography (PET/CT).

In the present disclosure, CMS with prominent biocompatibility is adopted as a carrier loaded with the therapeutic radionuclide and zirconium [⁸⁹Zr] and then dispersed in a special solution to obtain the suspension. The suspension can be relatively evenly distributed in tumor tissues after being administered through catheter intervention, injection, or the like, and the suspension can be distributed into various solid tumors including liver cancer, pancreatic cancer, kidney cancer, breast cancer, thyroid cancer, and bowel cancer to achieve the visualized brachytherapy.

The visualizable CMS of the present disclosure can be mainly used for the treatment of a solid tumor and the visualized treatment through PET/CT during a treatment process and can also be used as a therapeutic drug and an imaging drug. After the visualizable CMS enters a tumor through injection, intervention, or the like, the therapeutic radionuclide emits high-energy β rays to kill tumor cells, and positrons emitted by the zirconium [⁸⁹Zr] are collected through PET to achieve imaging, thereby realizing the visualized treatment of a tumor lesion.

Compared with the prior art, the present disclosure has the following beneficial effects.

1. A therapeutic radionuclide and an imaging radionuclide in different groups in the periodic table of elements usually have completely different chemical properties, and thus can hardly be stably and efficiently loaded on the same carrier. The preparation method of the present disclosure overcomes the above technical problem and realizes the simultaneous loading of a therapeutic radionuclide (any one selected from the group consisting of yttrium [⁹⁰Y], lutetium [¹⁷⁷Lu], holmium [¹⁶⁶Ho], samarium [¹⁵³Sm], and isotopes thereof) and an imaging radionuclide zirconium [⁸⁹Zr] on CMS. The radionuclides in the CMS product prepared in the present disclosure have a load rate of greater than 98% and a release rate of less than 0.1%.

2. The visualizable radioactive CMS suspension of the present disclosure can simultaneously realize the local radiotherapy and real-time imaging of a solid tumor lesion, thereby realizing the visualized treatment of a tumor. The present disclosure provides a new radioactive CMS product that integrates diagnosis and treatment. The visualizable CMS of the present disclosure is a brand-new radioactive CMS product loaded with both the therapeutic radionuclide and the imaging radionuclide zirconium [⁸⁹Zr], which has not been found in other documents and patents.

3. The visualizable CMS of the present disclosure exhibits a significant tumor treatment effect, can provide a high-quality PET/CT image during a treatment process, and has the potential to be developed into a therapeutic drug for treating solid tumors such as liver cancer, pancreatic cancer, kidney cancer, breast cancer, thyroid cancer, and bowel cancer.

### BRIEF DESCRIPTION OF THE DRAWINGS

FIG. 1 shows an SPECT image of a Beagle with a single administration of yttrium [⁹⁰Y] CMS through a hepatic artery in the background art;

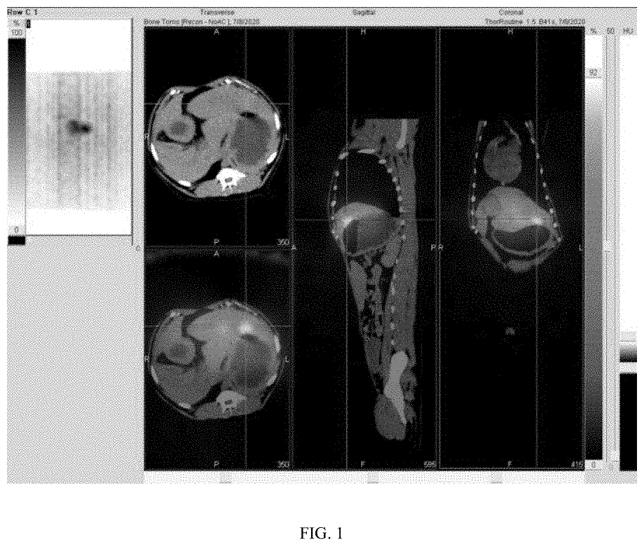

FIGS. 2A-2B show scanning electron microscopy (SEM) images of the visualizable radioactive CMS in Example 6;

FIGS. 3A-3D show local anatomy images of a tumor in a New Zealand rabbit treated for 15 d in the experimental group of Example 11; and

FIGS. 4A-4F show PET/CT maximum intensity projection (MIP) images of a New Zealand rabbit with a single administration of the visualizable CMS through a hepatic artery at different time points in Example 12.

### DETAILED DESCRIPTION OF THE EMBODIMENTS

In order to explain the technical solutions of the present disclosure, the technical solutions of the present disclosure will be further described below with reference to the accompanying drawings and examples. The implementations of the present disclosure include, but are not limited to, the following examples, which are intended to illustrate the present disclosure but are not intended to limit the protection scope of the present disclosure. Unless otherwise specified, the technical means used in the examples are conventional means known to those skilled in the art. Unless otherwise specified, the test methods in the following examples are conventional methods.

A labeling rate and a release rate of the radionuclide used in the examples of the present disclosure are tested as follows:

1. A radioactive CMS suspension that is to be visualized in an example is tested with an isotope activity meter (CRC-25R) for gross activity (A_{CMS suspension} or A₍₀₎).

2. 100 μL of the supernatant of the above suspension product is quantitatively determined with a high-purity germanium y spectrometer (GEM-C40-LB-C) to obtain an activity value of the supernatant (Aₛᵤₚₑᵣₙₐₜₐₙₜ).

3. The remaining suspension product in step 2 is placed in a centrifuge tube and centrifuged to obtain a CMS solid, which is then soaked in 10 mL of a sodium chloride injection with a mass percentage concentration of 0.9%. The resulting mixture is shaken in a 37±1° C. constant-temperature shaker for 24 h. 1 mL of the resulting supernatant is filtered to obtain a filtrate, and 100 μL of the filtrate is quantitatively tested with a high-purity germanium spectrometer (GEM-C40-LB-C) to obtain an activity value A₍ₜᵢ₎. Release rates of radionuclides from the visualizable radioactive CMS at different shaking time points are calculated.

(a) An adsorption rate (namely labeling rate) of the CMS for radionuclides is calculated according to formula (1):

$$Adsorption rate = 1 - \frac{{A}_{supernatant}}{{A}_{CMS suspension} \times k} \qquad ( 1 )$$

where Aₛᵤₚₑₘₐₜₐₙₜ represents the activity of the supernatant obtained after the CMS adsorbs the radionuclides;

A_{CMS suspension} represents the radionuclide activity of the CMS suspension (normalized to a measurement time point of an adsorption supernatant); and

k represents the calibration factor of the activity meter used to test the radionuclides under corresponding geometric conditions.

(b) At different time points, a release rate of the radionuclide CMS is calculated according to formula (2):

$$Release rate = \frac{{A}_{( {t}_{i} )}}{{A}_{( 0 )} {e}^{- λ t}} \qquad ( 2 )$$

where t represents a time interval from t=0 to a measurement time point;

A₍ₜᵢ₎ represents a radionuclide activity in a soaking solution at a time point t; and

A(0) represents a radionuclide activity of the visualizable radioactive CMS suspension at t=0.

Given a loss correction of each sampling, a total radionuclide activity A(tᵢ) of the soaking solution measured at the i-th time is calculated according to formula (3):

$$\text{?}$$

$$\text{?} \text{indicates text missing or illegible when filed}$$

where V represents the total volume of the soaking supernatant;

aᵢ and aⱼ represent radioactive concentrations of the soaking solution measured at the i-th and j-th times, respectively; and

tᵢ and tⱼ represent soaking times at the i-th and j-th sampling, respectively.

### Example 1

A visualizable radioactive CMS suspension is provided, where every 1 mL of the visualizable radioactive CMS suspension includes: CMS: 10 mg to 500 mg; a therapeutic radionuclide with an activity of 5 mCi to 500 mCi; radioactive zirconium [⁸⁹Zr] with an activity of 0.1 mCi to 100 mCi; a small organic molecule: 0 mg to 100 mg; and a first solution: 0.1 mL to 1.0 mL.

Specifically, the CMS may be a spherical or non-spherical carbon material rich in micropores and mesopores that is prepared by any method and may have a diameter of 0.05 μm to 1,000 μm and preferably 20 μm to 60 μm. Visualizable CMS products prepared from CMSs with different particle sizes can be used for the visualized treatment of different solid tumor lesions due to different administration routes and different distributions in tissues, organs, and lesions.

Specifically, the small organic molecule may be 5-SSA, 5-NSA, or a small molecule with a similar structure that is obtained through simple chemical modification.

Specifically, the first solution may include, but is not limited to, one or more selected from the group consisting of an ethanol solution, a PEG solution, and a glycerol solution; a water-soluble saccharide solution such as a glucose solution, a dextran solution, and a dextran solution; a water-soluble cellulose solution, such as a CMC-Na solution, a CEC-Na solution, and a HPC solution; a water-soluble starch solution, such as a HES solution and a CMS-Na solution; and another solution of a water-soluble small molecule or polymer with a similar structure.

Specifically, the therapeutic radionuclide may be any one selected from the group consisting of yttrium [⁹⁰Y], lutetium [¹⁷⁷Lu], holmium [¹⁶⁶Ho], samarium [¹⁵³Sm], and isotopes thereof. The therapeutic radionuclide and zirconium [⁸⁹Zr] may be water-soluble therapeutic radionuclide and zirconium [⁸⁹Zr] that are in different chemical forms and are prepared by a generator, a nuclear reactor, or any other common preparation method. A therapeutic radionuclide solution may have a radioactivity of 10 mCi to 100 Ci, and an imaging radioactive zirconium [⁸⁹Zr] solution may have a radioactivity of 0.1 mCi to 10 Ci.

### Example 2

A preparation method of a visualizable radioactive CMS suspension was provided, including the following steps:

A therapeutic radionuclide solution was thoroughly mixed with a small molecule aqueous solution, and the resulting mixed solution was allowed to stand for 2 min to 20 min.

The CMS was allowed to adsorb the therapeutic radionuclide in the mixed solution and then the resulting solution was allowed to stand for 2 min to 20 min.

The CMS adsorbing the therapeutic radionuclide was mixed with a sodium phosphate solution to allow a reaction for 2 min to 20 min, and the resulting system was washed and subjected to SLS to obtain a first intermediate.

The first intermediate was thoroughly mixed with a small organic molecule aqueous solution at a pH of 1 to 14 and preferably 3.5 to 6.5, and the resulting mixture was allowed to stand for 2 min to 20 min to obtain a first intermediate solution.

A radioactive zirconium [⁸⁹Zr] ion solution having a pH of 1 to 14 and preferably 3.5 to 7.5 was added to the first intermediate solution, and the resulting mixture was thoroughly mixed and allowed to stand for 2 min to 20 min to obtain a second intermediate.

The first solution was added to the second intermediate, and the resulting mixture was thoroughly mixed, dispensed, and subjected to moist-heat sterilization at 121° C. for 15 min.

### Example 3

A preparation method of the visualizable radioactive CMS suspension was provided, including the following steps:

CMS was allowed to adsorb a small organic molecule in a small organic molecule aqueous solution having a pH of 1 to 14 and preferably 3.5 to 6.5;

The CMS adsorbing the small organic molecule was allowed to adsorb the radioactive zirconium [⁸⁹Zr] to obtain a third intermediate.

The third intermediate was allowed to adsorb a therapeutic radionuclide in a mixed solution of a therapeutic radionuclide solution and a small organic molecule aqueous solution.

The third intermediate adsorbing the therapeutic radionuclide was mixed with a sodium phosphate solution to allow a reaction, and the resulting system was washed and subjected to SLS to obtain a fourth intermediate.

The first solution was added to the fourth intermediate, and the resulting mixture was thoroughly mixed, dispensed, and subjected to moist-heat sterilization at 121° C. for 15 min.

The visualizable radioactive CMS suspension can be used in the preparation of a drug for the visualized treatment of a solid tumor. In the present disclosure, the CMS with prominent biocompatibility is adopted as a carrier loaded with the therapeutic radionuclide and imaging nuclide zirconium [⁸⁹Zr] and then dispersed in a special solution to obtain the suspension. The suspension can be relatively evenly distributed in tumor tissues after being administered through catheter intervention, injection, or the like, and the suspension can be distributed into various solid tumors including liver cancer, pancreatic cancer, kidney cancer, breast cancer, thyroid cancer, and bowel cancer to achieve the visualized brachytherapy.

### Example 5

The visualizable radioactive CMS suspension can be used in the preparation of a drug for PET/CT. The visualized CMS of the present disclosure can be mainly used for the treatment of a solid tumor and the visualized treatment through PET/CT during a treatment process and can also be used as a therapeutic drug and an imaging drug. After the visualizable CMS enters a tumor through injection, intervention, or the like, the therapeutic radionuclide emits high-energy β rays to kill tumor cells, and positrons emitted by the zirconium [⁸⁹Zr] are collected through PET to achieve imaging, thereby realizing the visualized treatment of a tumor lesion.

### Example 6

A visualizable radioactive CMS suspension was prepared through the following process:

A radioactive yttrium [⁹⁰Y] solution with yttrium [⁸⁹Y] and a 5-SSA solution having a pH of 4.5 were mixed according to a yttrium/5-SSA molar ratio of 1:(1-100), and the resulting mixture was thoroughly shaken and then allowed to stand for 2 min to 20 min to obtain a mixed solution.

0.01 g to 100 g of CMS was added to the mixed solution for adsorbing the radioactive yttrium [⁹⁰Y], and the resulting mixture was thoroughly shaken and allowed to stand for 2 min to 20 min.

The CMS adsorbing the radioactive yttrium [⁹⁰Y] was mixed with a sodium phosphate solution having a pH of 6.5 and a molar concentration of 1% to allow a reaction, and the resulting reaction system was washed and subjected to SLS to obtain a first intermediate.

The first intermediate was mixed with a 5-SSA aqueous solution that included 0.01 mg to 10 mg of 5-SSA and had a pH of 4.5, and the resulting mixture was thoroughly shaken and allowed to stand for 2 min to 20 min to obtain a first intermediate solution.

A zirconium [⁸⁹Zr] solution having a pH of 4.0 was added to the first intermediate solution, and the resulting mixture was thoroughly shaken and allowed to stand for 2 min to 20 min to obtain a second intermediate. The supernatant was tested for zirconium [⁸⁹Zr] radioactivity, and the adsorption rate for the radionuclide was calculated to be 99.1%.

1 mL to 100 mL of a CMC-Na solution with a molar concentration of 0.2% was added to the second intermediate, and the resulting mixture was thoroughly mixed, dispensed into vials, and sterilized at 121° C. for 15 min to obtain the visualizable radioactive CMS suspension for injection.

The radionuclide in the sample of this example had an adsorption rate of 99.6% and a release rate of 0.006%.

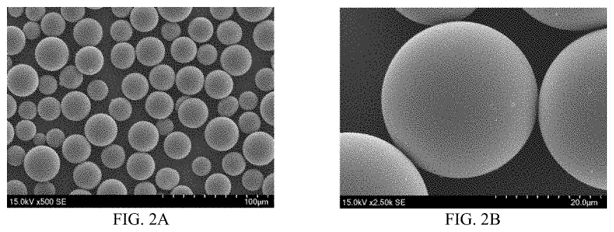

An SEM image of the prepared visualizable radioactive CMS was shown in FIGS. 2A-2B, and it can be seen that the CMS had a particle size mainly of 20 μm to 45 μm and a smooth surface.

### Example 7

A visualizable radioactive CMS suspension was prepared through the following process:

0.01 g to 100 g of CMS was added to a 5-SSA aqueous solution that included 0.01 mg to 10 mg of 5-SSA and had a pH of 4.0 to adsorb the 5-SSA, and the resulting mixture was thoroughly shaken and allowed to stand for 2 min to 20 min.

The CMS adsorbing 5-SSA was added to a 0.1 Ci to 10 Ci zirconium [⁸⁹Zr] solution having a pH of 5.0, and the resulting mixture was thoroughly shaken and then allowed to stand for 2 min to 20 min to obtain a third intermediate. The supernatant was tested for zirconium [⁸⁹Zr] radioactivity, and the adsorption rate for the radionuclide was calculated to be 99.3%. A radioactive yttrium solution with yttrium [⁹⁰Y] and yttrium [⁸⁹Y] and a 5-SSA solution were mixed according to a yttrium/5-SSA molar ratio of 1:(1-100), and the resulting mixture was thoroughly shaken and then allowed to stand for 2 min to 20 min to obtain a mixed solution.

The third intermediate was added to the mixed solution of the radioactive yttrium [⁹⁰Y] solution and the 5-SSA solution for adsorbing the radioactive yttrium [⁹⁰Y], and the resulting mixture was thoroughly shaken and allowed to stand for 2 min to 20 min.

The third intermediate adsorbing the radioactive yttrium [⁹⁰Y] was mixed with a sodium phosphate solution having a pH of 6.5 and a molar concentration of 1% to allow a reaction, and the resulting reaction system was washed and subjected to SLS to obtain a fourth intermediate.

1 mL to 100 mL of an HES solution with a molar concentration of 6% was added to the fourth intermediate, and the resulting mixture was thoroughly mixed, dispensed into vials, and sterilized at 121° C. for 15 min to obtain the visualizable radioactive CMS suspension for injection.

The radionuclide in the sample of this example had an adsorption rate of 99.8% and a release rate of 0.023%.

### Example 8

A visualizable radioactive CMS suspension was prepared through the following process:

A 400 mCi lutetium [¹⁷⁵Lu] and lutetium [¹⁷⁷Lu]-containing radioactive solution was mixed with a 5-SSA solution having a pH of 3.5 according to a lutetium/5-SSA molar ratio of 1:4, and the resulting mixture was thoroughly shaken and allowed to stand for 10 min to obtain a mixed solution.

1.0 g of CMS was added to the mixed solution for adsorbing the radioactive lutetium [¹⁷⁵Lu] and lutetium [¹⁷⁷Lu], and the resulting mixture was thoroughly shaken and then allowed to stand for 2 min to 20 min. The supernatant was tested for lutetium [¹⁷⁷Lu] radioactivity, and the adsorption rate for the radionuclide was calculated to be 98.8%. The CMS adsorbing the radioactive lutetium [¹⁷⁵Lu] and lutetium [¹⁷⁷Lu] was mixed with a sodium phosphate solution having a pH of 6.5 and a molar concentration of 1% to allow a reaction, and the resulting reaction system was washed and subjected to SLS to obtain a first intermediate.

The first intermediate was mixed with a 5-SSA aqueous solution that included 3 mg of 5-SSA and had a pH of 3.5, and the resulting mixture was thoroughly shaken and allowed to stand for 2 min to obtain a first intermediate solution.

A 10 mCi zirconium [⁸⁹Zr] solution having a pH of 4.5 was added to the first intermediate solution, and the resulting mixture was thoroughly shaken and allowed to stand for 20 min to obtain a second intermediate.

5 mL of a CMC-Na solution with a molar concentration of 0.2% was added to the second intermediate, and the resulting mixture was thoroughly mixed, dispensed into 20 vials, and sterilized at 121° C. for 15 min to obtain the visualizable radioactive CMS suspension for injection.

The radionuclide in the sample of this example had an adsorption rate of 99.9% and a release rate of 0.056%.

A visualizable radioactive CMS suspension was prepared through the following process:

An 800 mCi holmium [¹⁶⁵Ho] and holmium [166Ho] containing radioactive solution was mixed with a 5-SSA solution having a pH of 6.5 according to a holmium/5-SSA molar ratio of 1:3, and the resulting mixture was thoroughly shaken and allowed to stand for 5 min to obtain a mixed solution.

1.0 g of CMS was added to the mixed solution for adsorbing the radioactive holmium [¹⁶⁵Ho] and holmium [¹⁶⁶Ho] and the resulting mixture was thoroughly shaken and allowed to stand for 5 min.

The CMS adsorbing the radioactive holmium [¹⁶⁵Ho] and holmium [¹⁶⁶Ho] was mixed with a sodium phosphate solution having a pH of 6.5 and a molar concentration of 1% to allow a reaction, and the resulting reaction system was washed and subjected to SLS to obtain a first intermediate.

The first intermediate was mixed with a 5-SSA aqueous solution that included 1 mg of 5-SSA and had a pH of 6.5, and the resulting mixture was thoroughly shaken and allowed to stand for 6 min to obtain a first intermediate solution.

A 5 mCi zirconium [⁸⁹Zr] solution having a pH of 4.5 was added to the first intermediate solution, and the resulting mixture was thoroughly shaken and allowed to stand for 20 min to obtain a second intermediate. The supernatant was tested for zirconium [⁸⁹Zr] radioactivity, and the adsorption rate for the radionuclide was calculated to be 99.8%.

5 mL of a CMC-Na solution with a molar concentration of 0.2% was added to the second intermediate, and the resulting mixture was thoroughly mixed, dispensed into 20 vials, and sterilized at 121° C. for 15 min to obtain the visualizable radioactive CMS suspension for injection.

The radionuclide in the sample of this example had an adsorption rate of 99.5% and a release rate of 0.034%.

### Example 10

A visualizable radioactive CMS suspension was prepared through the following process:

A 1,000 mCi samarium [¹⁵⁰Sm] and samarium [¹⁵³Sm]-containing radioactive solution was mixed with a 5-SSA solution having a pH of 5.5 according to a samarium/5-SSA molar ratio of 1:8, and the resulting mixture was thoroughly shaken and allowed to stand for 15 min to obtain a mixed solution.

1.0 g of CMS was added to the mixed solution for adsorbing the radioactive samarium [¹⁵⁰Sm] and samarium [¹⁵³Sm], and the resulting mixture was thoroughly shaken and allowed to stand for 5 min;

The CMS adsorbing the radioactive samarium [¹⁵⁰Sm] and samarium [¹⁵³Sm] was mixed with a sodium phosphate solution having a pH of 6.5 and a molar concentration of 1% to allow a reaction, and the resulting reaction system was washed and subjected to SLS to obtain a first intermediate.

The first intermediate was mixed with a 5-SSA aqueous solution that included 1 mg of 5-SSA and had a pH value of 5.5, and the resulting mixture was thoroughly shaken and allowed to stand for 6 min to obtain a first intermediate solution.

An 8 mCi zirconium [⁸⁹Zr] solution having a pH of 4.5 was added to the first intermediate solution, and the resulting mixture was thoroughly shaken and allowed to stand for 20 min to obtain a second intermediate. The supernatant was tested for zirconium [⁸⁹Zr] radioactivity, and the adsorption rate for the radionuclide was calculated to be 99.9%;

5 mL of a CMC-Na solution with a molar concentration of 0.2% was added to the second intermediate, and the resulting mixture was thoroughly mixed, dispensed into 20 vials, and sterilized at 121° C. for 15 min to obtain the visualizable radioactive CMS suspension for injection.

The radionuclide in the sample of this example had an adsorption rate of 99.3% and a release rate of 0.018%.

### Example 11

The visualizable CMS suspension of Example 7 of the present disclosure was in situ injected into a New Zealand rabbit surface xenograft model for efficacy evaluation:

Experimental group: 4 New Zealand rabbits: The rabbits were administered with the visualizable CMS suspension at a dose of 50 mg/rabbit to 100 mg/rabbit, in which a yttrium [⁸⁹Y] activity was 1.3 mCi to 2.5 mCi and a zirconium [⁸⁹Zr] activity was 0.13 mCi to 0.25 mCi. Each rabbit was administered with 80 mg of the suspension on average, and an administration volume was 5% of the tumor volume.

Control group: 2 tumor-bearing New Zealand rabbits: The rabbits were administered with an equal volume of normal saline (NS).

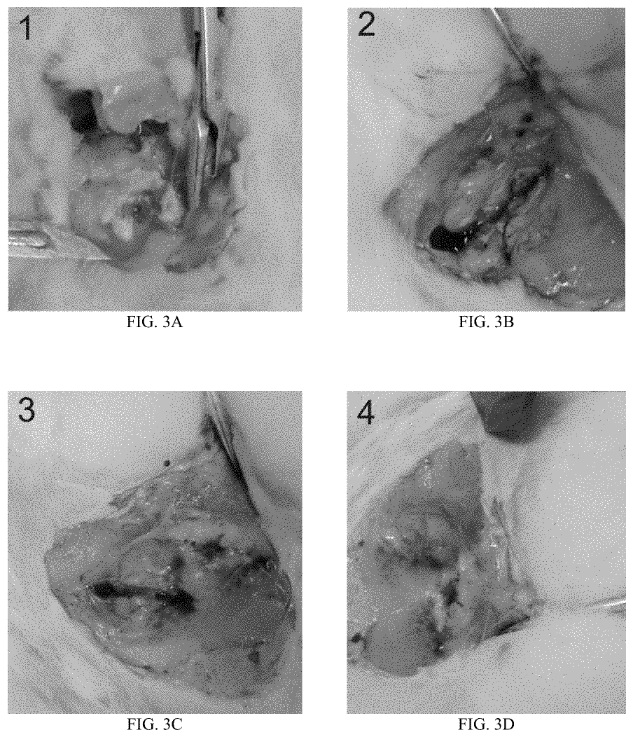

The ultrasonic observation and size measurements were collected after 5 d of treatment, and results showed that the tumors in the 4 New Zealand rabbits of the experimental group did not grow, but tumors in the 2 New Zealand rabbits of the control group grew significantly. The New Zealand rabbits were subjected to imaging of local anatomy after 15 d of treatment, and local anatomy images of the tumors of the 4 rabbits in the experimental group are shown in FIGS. 3A-3D. Through the images, it was observed that black CMS particles were visible locally at administration sites of tumors of the 4 New Zealand rabbits, and a tumor tissue near the visualizable CMS turned gray-brown and underwent obvious fibrotic necrosis. It indicates that the visualizable CMS suspensions prepared in the examples of the present disclosure exhibit excellent anti-tumor activity.

### Example 12

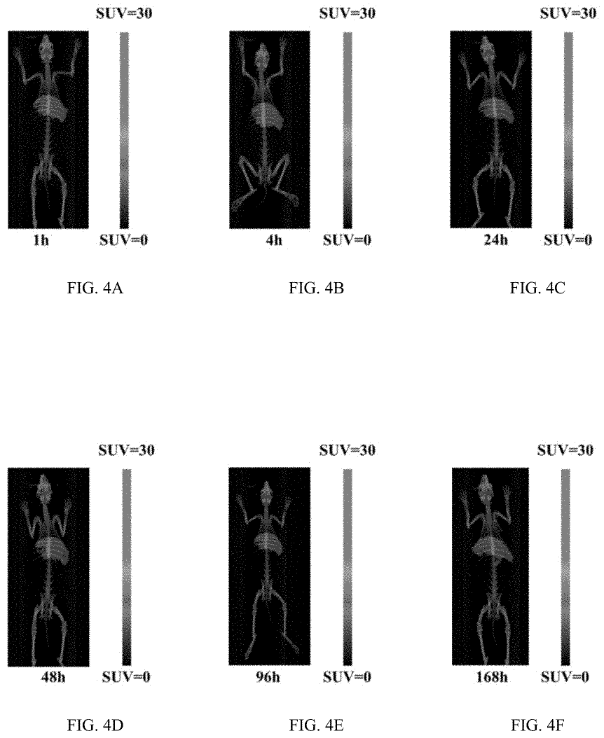

The visualizable CMS suspension of Example 7 of the present disclosure was administered to New Zealand rabbits through hepatic artery cannulation, and the distribution of the visualizable CMS suspension in animals was investigated: Each New Zealand rabbit was administered with about 20 mg of the suspension, and the whole body of the rabbit was scanned using PET at 1 h, 4 h, 24 h, 48 h, 96 h, and 168 h after the administration of the visualizable radioactive CMS suspension. With the animal in a fixed position, CT scanning was conducted before/after the PET scanning. Results are shown in FIGS. 4A-4F.

After the PET/CT scanning was completed, image reconstruction was conducted, and the PMOD software was used to process the images and data. The brain, heart, liver, spleen, lung, kidney, stomach, bone, muscle, and the like were delineated as regions of interest (ROIs). The radioactivity concentrations (namely a radioactivity value per unit volume) of the ROIs were determined, and then the decay correction was determined for an activity at each time point. Results are shown in Table 1. The following formulas were adopted:

$$Corrected activity = initial activity \times {0.5}^{\hat{}} \frac{( correction time point - initial time point )}{half - life of nuc lide}$$

$$Radioactive concentration of tissue ( μ gEqu . / g ) = \frac{Radioactivity concentration of ROI ( μ Ci / g )}{specific activity ( μ Ci / g )}$$

TABLE 1
Radioactive concentrations of each tissue in New Zealand rabbits with a single
administration of the visualizable CMS through a hepatic artery at different time points

| Time point/h | | | | | | |
|---|---|---|---|---|---|---|
| Tissue | 1 | 4 | 24 | 48 | 96 | 168 |
| Brain | 0.02 ± 0.01 | 0.04 ± 0.03 | 0.13 ± 0.03 | 0.14 ± 0.02 | 0.19 ± 0.11 | 0.11 ± 0.01 |
| Heart | 0.14 ± 0.02 | 0.17 ± 0.05 | 0.10 ± 0.07 | 0.16 ± 0.01 | 0.10 ± 0.02 | 0.09 ± 0.01 |
| Left lung | 0.42 ± 0.26 | 0,50 ± 0.39 | 0.40 ± 0.33 | 1.45 ± 0.73 | 1.14 ± 0.79 | 0.47 ± 0.34 |
| Right lung | 0.46 ± 0.13 | 0.25 ± 0.06 | 0,35 ± 0.05 | 2.21 ± 0.24 | 0.65 ± 0.14 | 0,29 ± 0.03 |
| Left liver | 90.49 ± 21.72 | 82.38 ± 42.46 | 89.49 ± 64.34 | 92.15 ± 61.28 | 100.90 ± 65.23 | 62.62 ± 32.14 |
| Right liver | 126.29 ± 37.63 | 109.20 ± 66.47 | 110.38 ± 75.38 | 121.40 ± 76.37 | 100.08 ± 46.76 | 98.50 ± 49.47 |
| Kidney | 0.12 ± 0.04 | 0.13 ± 0.00 | 0.18 ± 0.11 | 0.24 ± 0.10 | 0.15 ± 0.09 | 0.19 ± 0.05 |
| Spleen | 0.05 ± 0.03 | 0.08 ± 0.08 | 0.07 ± 0.06 | 0.13 ± 0.00 | 0.08 ± 0.05 | 0.10 ± 0.10 |
| Bone joint | 0.00 ± 0.00 | 0.00 ± 0.00 | 0.00 ± 0.01 | 0.01 ± 0.01 | 0.02 ± 0.00 | 0.01 ± 0.00 |
| Shinbone | 0.00 ± 0.00 | 0.00 ± 0.00 | 0.00 ± 0.01 | 0.01 ± 0.01 | 0.02 ± 0.00 | 0.01 ± 0.00 |
| Muscle | 0.00 ± 0.00 | 0.01 ± 0.01 | 0.01 ± 0.00 | 0.01 ± 0.00 | 0.01 ± 0.01 | 0.02 ± 0.00 |
| Stomach | 0.15 ± 0.09 | 0.20 ± 0.05 | 0,20 ± 0.08 | 0.25 ± 0.15 | 0.20 ± 0.19 | 0.33 ± 0.22 |

FIGS. 3A-3D show clear images illustrating the distribution of the visualized CMSs in animals. It can be seen from the figure and the data of uptake in various organs and tissues in Table 1 that the visualized CMSs are concentrated in the liver with a proportion of more than 99%, and a small amount of the CMSs are distributed in other organs and tissues.

It can be seen from the results in FIGS. 2A-2B, FIGS. 3A-3D, FIGS. 4A-4F, and Table 1 that the radionuclide in the CMS product prepared by the present disclosure has a load rate of greater than 98% and a release rate of lower than 0.1%. The present disclosure achieves both the local radiotherapy and the real-time imaging of a solid tumor lesion, that is, the present disclosure realizes the visualized treatment of a tumor.

In summary, any combination of various different examples of the present disclosure without departing from the idea of the present disclosure shall be regarded as part of the content disclosed in the present disclosure. Various simple modifications made to the technical solutions and any combination of different examples without departing from the idea of the present disclosure should all fall within the protection scope of the present disclosure.

What is claimed is:

1. A visualizable radioactive carbon microsphere (CMS) suspension, wherein every 1 mL of the visualizable radioactive CMS suspension comprises:
10 mg to 500 mg of CMS,
a therapeutic radionuclide with an activity of 5 mCi to 500 mCi,
an imaging radionuclide with an activity of 0.1 mCi to 100 mCi,
0 mg to 100 mg of an organic molecule, and
0.1 mL to 1.0 mL of a first solution.

2. The visualizable radioactive CMS suspension according to claim 1,
wherein the CMS is a spherical or non-spherical carbon material with a plurality of micropores and mesopores,
and
wherein the CMS has a diameter of 0.05 μm to 1,000 μm.

3. The visualizable radioactive CMS suspension according to claim 1, wherein the organic molecule is 5-sulfosalicylic acid (5-SSA), 5-nitrosalicylic acid (5-NSA), or a molecule modified to have a first structure similar to the 5-SSA or the 5-NSA.

4. The visualizable radioactive CMS suspension according to claim 1,
wherein the therapeutic radionuclide is one selected from the group consisting of yttrium [⁹⁰Y], lutetium [¹⁷⁷Lu], holmium [¹⁶⁶Ho] samarium [¹⁵³Sm], an isotope of the yttrium [⁹⁰Y], an isotope of the lutetium [¹⁷⁷Lu], an isotope of the holmium [¹⁶⁶Ho], and an isotope of the samarium [¹⁵³Sm]; and
wherein the imaging radionuclide is zirconium [⁸⁹Zr].

5. The visualizable radioactive CMS suspension according to claim 1,
wherein the first solution comprises an ethanol solution, a polyethylene glycol (PEG) solution, a glycerol solution, a water-soluble saccharide solution, a water-soluble cellulose solution, a water-soluble starch solution, and a solution of a water-soluble polymer or molecule;
wherein the water-soluble saccharide solution comprises a glucose solution and a dextran solution; the water-soluble cellulose solution comprises a sodium carboxymethyl cellulose (CMC-Na) solution, a sodium carboxyethyl cellulose (CEC-Na) solution, and a hydroxypropyl cellulose (HPC) solution; and the water-soluble starch solution comprises a hydroxyethyl starch (HES) solution and a sodium carboxymethyl starch (CMS-Na) solution; and
wherein the water-soluble polymer or molecule is a polymer or molecule with a second structure similar to ethanol, polyethylene glycol, glycerol, glucose, dextran, sodium carboxymethyl cellulose, sodium carboxyethyl cellulose, hydroxypropyl cellulose, hydroxyethyl starch, and sodium carboxymethyl starch.

6. A method of preparing the visualizable radioactive CMS suspension according to claim 1 comprising the following steps:
mixing a therapeutic radionuclide solution with an organic molecule aqueous solution having a first pH value to obtain a mixed solution;
allowing the CMS to adsorb the therapeutic radionuclide in the mixed solution;
mixing the CMS adsorbing the therapeutic radionuclide with a sodium phosphate solution to allow a reaction, washing a resulting solution, and conducting a solid-liquid separation (SLS) on a washed solution to obtain a first intermediate;
mixing the first intermediate with the organic molecule aqueous solution having the first pH value to obtain a first intermediate solution;
adding a radioactive zirconium [⁸⁹Zr] ion solution having a second pH value to the first intermediate solution to obtain a second intermediate; and
adding the first solution to the second intermediate, and mixing and sterilizing a resulting mixture.

7. A method of preparing the visualizable radioactive CMS suspension according to claim 1 comprising the following steps:
allowing the CMS to adsorb the organic molecule in an organic molecule aqueous solution having a first pH value;
allowing the CMS adsorbing the organic molecule to adsorb a radioactive zirconium [⁸⁹Zr] to obtain a third intermediate;
allowing the third intermediate to adsorb the therapeutic radionuclide in a mixed solution of a therapeutic radionuclide ion solution and the organic molecule aqueous solution;
mixing the third intermediate adsorbing the therapeutic radionuclide with a sodium phosphate solution to allow a reaction, washing a resulting solution, and conducting an SLS on a washed solution to obtain a fourth intermediate; and
adding the first solution to the fourth intermediate, and mixing and sterilizing a resulting mixture.

8. The method according to claim 6, wherein the first pH value is from 1 to 14, and the second pH value is from 1 to 14.

9. The visualizable radioactive CMS suspension prepared by the method according to claim 6.

10. A method of use of the visualizable radioactive CMS suspension according to claim 1 in a preparation of a drug for a verbalizable treatment of a tumor, wherein the tumor comprises a liver cancer, a pancreatic cancer, a kidney cancer, a breast cancer, a thyroid cancer, a bowel solid tumor, and a bone tumor.

11. The method according to claim 7, wherein the first pH value is from 1 to 14, and the second pH value is from 1 to 14.

12. The visualizable radioactive CMS suspension prepared by the method according to claim 7.

13. The visualizable radioactive CMS suspension prepared by the method according to claim 8.

14. The visualizable radioactive CMS suspension prepared by the method according to claim 11.

15. The method according to 10,
wherein the CMS is a spherical or non-spherical carbon material with a plurality of micropores and mesopores, and
wherein the CMS has a diameter of 0.05 μm to 1,000 μm.

16. The method according to 10,
wherein the organic molecule is 5-sulfosalicylic acid (5-SSA), 5-nitrosalicylic acid (5-NSA), or a molecule modified to have a structure similar to the 5-SSA or the 5-NSA.

17. The method according to 10,
wherein the therapeutic radionuclide is one selected from the group consisting of yttrium [⁹⁰Y], lutetium [¹⁷⁷Lu], holmium [¹⁶⁶Ho] samarium [¹⁵³Sm], an isotope of the yttrium [⁹⁰Y], an isotope of the lutetium [¹⁷⁷Lu], an isotope of the holmium [¹⁶⁶Ho], and an isotope of the samarium [¹⁵³Sm]; and
wherein the imaging radionuclide is zirconium [⁸⁹Zr].

18. The method according to 10,
wherein the first solution comprises an ethanol solution, a polyethylene glycol (PEG) solution, a glycerol solution, a water-soluble saccharide solution, a water-soluble cellulose solution, and a water-soluble starch solution, and
wherein the water-soluble saccharide solution comprises a glucose solution and a dextran solution; the water-soluble cellulose solution comprises a sodium carboxymethyl cellulose (CMC-Na) solution, a sodium carboxyethyl cellulose (CEC-Na) solution, and a hydroxypropyl cellulose (HPC) solution; and the water-soluble starch solution comprises a hydroxyethyl starch (HES) solution and a sodium carboxymethyl starch (CMS-Na) solution.

19. The method according to claim 6, wherein the therapeutic radionuclide is one selected from the group consisting of yttrium [⁹⁰Y], lutetium [¹⁷⁷Lu], holmium [¹⁶⁶Ho], samarium [¹⁵³Sm], an isotope of the yttrium [⁹⁰Y], an isotope of the lutetium [¹⁷⁷Lu], an isotope of the holmium [¹⁶⁶Ho], and an isotope of the samarium [¹⁵³Sm]; and
wherein the imaging radionuclide is zirconium [⁸⁹Zr].

20. The method according to of claim 7,
wherein the therapeutic radionuclide is one selected from the group consisting of yttrium [⁹⁰Y], lutetium [¹⁷⁷Lu], holmium [¹⁶⁶Ho], samarium [¹⁵³Sm], an isotope of the yttrium [⁹⁰Y], an isotope of the lutetium [¹⁷⁷Lu], an isotope of the holmium [¹⁶⁶Ho], and an isotope of the samarium [¹⁵³Sm]; and
wherein the imaging radionuclide is zirconium [⁸⁹Zr].
